(19) United States
(12) Patent Application Publication
(10) Pub. No.: US 2021/0338853 A1
(43) Pub. Date: Nov. 4, 2021

(54) PORTABLE FINGER DISINFECTION APPARATUS

(71) Applicant: INTERMAN Corporation, Kagoshima-shi (JP)

(72) Inventor: Shigeki UETABIRA, Kagoshima-shi (JP)

(73) Assignee: INTERMAN Corporation, Kagoshima-shi (JP)

(21) Appl. No.: 17/241,590

(22) Filed: Apr. 27, 2021

(30) Foreign Application Priority Data
May 2, 2020 (JP) 2020-081617

(51) Int. Cl. A61L 2/00; A61L 2/26

(52) U.S. Cl. CPC A61L 2/0047; A61L 2/0088; A61L 2202/16; A61L 2202/11; A61L 2202/122; A61L 2/26

(57) ABSTRACT

A portable finger disinfection apparatus is described which can be freely carried by each user so that fingers can easily be disinfected anywhere. The portable finger disinfection apparatus is provided with a main body having five holes into which can be inserted the tip portions of the forefinger, middle finger, annular finger, pinky finger and thumb of the user respectively, and a disinfection mechanism installed in the main body and capable of disinfecting the fingers inserted into the holes. For example, the disinfection mechanism is an ultraviolet ray generation device which can irradiate the finger inserted into the hole with ultraviolet rays. Alternatively, the disinfection mechanism is a disinfectant supply device which can supply the holes with a disinfectant.

### CROSS-REFERENCE TO RELATED APPLICATIONS

The present application claims priority under 35 U.S.C. § 119 to Japanese Patent Application No. P2020-081617, filed on May 2, 2020 including description, claims, drawings, and abstract. The contents of this application are herein incorporated by reference in their entirety.

### BACKGROUND OF THE INVENTION

### Field of the Invention

The present invention relates to a portable finger disinfection apparatus with which it is possible to disinfect fingers without difficulty.

### Description of Related Art

The new coronavirus has occurred a pandemic and let people know how the densely populated, high-speed and volume transportation society in the 21st century is vulnerable to an infectious disease.

In such a situation, everything around us looks an infectious agent so that daily life becomes confining. In fact, it seems necessarily to doubt everything in an infection spread situation.

To physically defend ourselves, complete disinfection is important. The way of disinfection is, for example, hand washing, the use of disinfectant, ultraviolet sterilization, or the like. Japanese Patent Published Application No. Hei8-107924 describes an example of a conventional air finger sterilization apparatus. This air finger sterilization apparatus sterilizes fingers and air by the use of ultraviolet rays.

However, while hand washing is effective, there is a problem that it is not always correctly performed. Also, the air finger sterilization apparatus described in Japanese Patent Published Application No. Hei8-107924 requires an appropriate place where this apparatus can be installed, and it is troublesome to go to the place for using this apparatus.

Taking into consideration the above circumstances, it is an object of the present invention therefore to provide a portable finger disinfection apparatus which can be freely carried by each user so that fingers can easily be disinfected anywhere.

### SUMMARY OF THE INVENTION

To achieve at least one of the above-mentioned objects, reflecting one aspect of the present invention, a portable finger disinfection apparatus comprises a main body having a support structure which supports a finger of a user; a disinfection mechanism installed in the main body and capable of disinfecting the finger supported by the support structure.

In accordance with one embodiment of the portable finger disinfection apparatus of the present invention, it is preferred that the support structure is a hole formed on the main body into which a tip portion of the finger of the user can be inserted.

Furthermore, in accordance with one embodiment of the portable finger disinfection apparatus of the present invention, it is preferred that the disinfection mechanism is an ultraviolet ray generation device which can irradiate the finger inserted into the hole with ultraviolet rays.

Particularly, in accordance with one embodiment of the portable finger disinfection apparatus of the present invention, it is preferred that the ultraviolet ray generation device is an ultraviolet ray LED.

Furthermore, in accordance with one embodiment of the portable finger disinfection apparatus of the present invention, it is preferred that the disinfection mechanism is a disinfectant supply device which can supply the hole with a disinfectant.

Particularly, in accordance with one embodiment of the portable finger disinfection apparatus of the present invention, it is preferred that the inside of the main body is filled with a spongy absorbable material which is exposed to the hole and in which the disinfectant supply device is installed, wherein the disinfectant supply device comprises a flexible disinfectant tank which stores the disinfectant and a disinfectant supply port for supplying the disinfectant to the hole, and wherein when the user inserts the tip portion of the finger into the hole and applies a pressure to the spongy absorbable material, the flexible disinfectant tank is deformed by the pressure to eject the disinfectant through the disinfectant supply port so that the spongy absorbable material becomes wet with the disinfectant to disinfect the tip portion of the finger tip inserted into the hole.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is a perspective view for showing a portable finger disinfection apparatus 10 in accordance with an embodiment 1 of the present invention.

FIG. 2 is a perspective view for showing the usage state of the portable finger disinfection apparatus 10 in accordance with the embodiment 1 of the present invention.

FIG. 3 is a perspective view for showing a modification example of the portable finger disinfection apparatus 10 in accordance with the embodiment 1 of the present invention.

FIG. 4 is a perspective view for showing the usage state of the modification example of the portable finger disinfection apparatus 10 in accordance with the embodiment 1 of the present invention.

FIG. 5 is a perspective view for showing a portable finger disinfection apparatus 30 in accordance with an embodiment 2 of the present invention.

FIG. 6 is a perspective view for showing the usage state of the portable finger disinfection apparatus 30 in accordance with the embodiment 2 of the present invention.

FIG. 7 is a perspective view for showing a portable finger disinfection apparatus 50 in accordance with an embodiment 3 of the present invention.

FIG. 8 is a perspective view for showing another example of the portable finger disinfection apparatus in accordance with the embodiment 3 of the present invention.

FIG. 9 is a perspective view for showing a modification example of the portable finger disinfection apparatus in accordance with the embodiment 3 of the present invention.

FIG. 10 is a perspective view for showing a portable finger disinfection apparatus 80 in accordance with an embodiment 4 of the present invention.

FIG. 11 is a perspective view for showing another example of the portable finger disinfection apparatus in accordance with the embodiment 4 of the present invention.

FIG. 12 is a view for showing a portable finger disinfection apparatus 90 in accordance with an embodiment 5 of the present invention.

FIG. 13 is a perspective view for showing the usage state of the portable finger disinfection apparatus 90 in accordance with the embodiment 5 of the present invention.

FIG. 14 is a cross sectional view for showing a liquid crystal back light device implemented within a smartphone which functions also as a portable finger disinfection apparatus according to an embodiment 6 of the present invention.

### DETAILED DESCRIPTION OF THE PREFERRED EMBODIMENT

### Embodiment 1

In what follows, a portable finger disinfection apparatus in accordance with an embodiment of the present invention will be explained with reference to the accompanying drawings. This portable finger disinfection apparatus makes use of the sterilizing effects of ultraviolet rays.

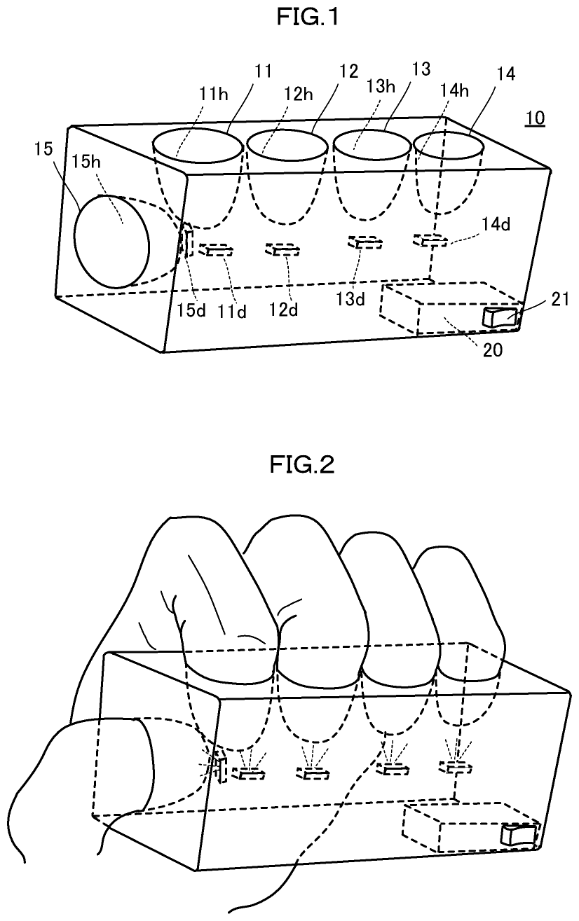

FIG. 1 is a perspective view for showing the portable finger disinfection apparatus 10 in accordance with the embodiment 1 of the present invention. The portable finger disinfection apparatus 10 comprises an approximately rectangular parallelepiped main body which is provided with four holes 11, 12, 13 and 14 on its upper surface and one hole 15 on its side surface.

The inner space of the rectangular parallelepiped portable finger disinfection apparatus 10 is filled with a spongy material having an ultraviolet ray transparent property except for the holes 11, 12, 13, 14 and 15 which are provided respectively with concaves 11h, 12h, 13h, 14h and 15h into which the forefinger, middle finger, annular finger, pinky finger and thumb can be just fitted respectively. The material transmitting ultraviolet rays is, for example, cellophane, polypropylene or the like.

Also, as an ultraviolet ray generation device capable of emitting ultraviolet rays, ultraviolet

LEDs 11d, 12d, 13d, 14d and 15d are arranged in the spongy material to face the bottoms of the concaves 11h, 12h, 13h, 14h and 15h respectively. Furthermore, a circuit device 20 is provided on the bottom portion of the rectangular parallelepiped main body of the portable finger disinfection apparatus 10. The circuit device 20 is implemented with a battery, a timer and the like.

The ultraviolet LEDs 11d, 12d, 13d, 14d and 15d are supplied with electric power from the circuit device 20 through wiring (not shown in the figure) to emit ultraviolet rays. The circuit device 20 is provided with a switch 21 which is exposed to the outside of the main body of the portable finger disinfection apparatus 10 and operated to turn on/off light emission. Furthermore, the circuit device 20 is provided with a USB port (not shown in the figure) for charging the battery. For example, even when the battery ran out of power during going out, power supply is possible from a smartphone or the like carried by the user through the USB port. Incidentally, the outer case of the portable finger disinfection apparatus 10 is made of a plastic and provided with a thin aluminum foil which is stuck to the inside surface of the outer case for reflecting ultraviolet rays.

FIG. 2 is a perspective view for showing the usage state of the portable finger disinfection apparatus 10. Namely, when the switch 21 is turned on, the ultraviolet LEDs 11d, 12d, 13d, 14d and 15d emit ultraviolet rays to the bottom portions of the concaves 11h, 12h, 13h, 14h and 15h into which the tip portions of the forefinger, middle finger, annular finger, pinky finger and thumb (the parts above the first joints) are inserted respectively, so that the ultraviolet rays illuminate and disinfect the tip portions of these fingers.

It is said in general that ultraviolet ray irradiation for a long time is harmful to human bodies. For example, if the portable finger disinfection apparatus is kept in a pocket together with fingers being inserted into this portable finger disinfection apparatus, there is a fear that the fingers are substantially damaged. Accordingly, it is desired to start a timer when starting irradiation, and automatically stop power supply to the ultraviolet LEDs when a predetermined time for example 5 minutes elapses.

The inner space of the portable finger disinfection apparatus 10 of the above embodiment is filled with the spongy material transmitting ultraviolet rays. The spongy material is provided for the purpose of fixing the positions of fingers when the fingers are inserted into the portable finger disinfection apparatus 10. However, as long as serving the same functionality, any alternative can be used such as a sack-like polypropylene finger rest. However, since such a spongy material installed in the portable finger disinfection apparatus 10 is porous to transmit and scatter ultraviolet rays, it is possible to evenly and effectively disinfect the entirety of fingertips with ultraviolet rays.

Furthermore, depending upon the arrangement of the ultraviolet LEDs, it may be possible to omit the spongy material and provide only the holes 11, 12, 13, 14 and 15 to leave the inside of the portable finger disinfection apparatus 10 as a hollow space. Even in such a case, the disinfection of fingers is possible without problem. Anyway, it is desirable not to allow leakage of ultraviolet rays for the purpose of avoiding damages to eyes.

In FIG. 1 and FIG. 2, the holes 11, 12, 13, 14 and 15, the concaves 11h, 12h, 13h, 14h and 15h, and the ultraviolet LEDs 11d, 12d, 13d, 14d and 15d are symmetrically arranged in the front and back direction as seen from the figures. In other words, it will be understood that this portable finger disinfection apparatus 10 can be used with either of the left and right hands in the same manner.

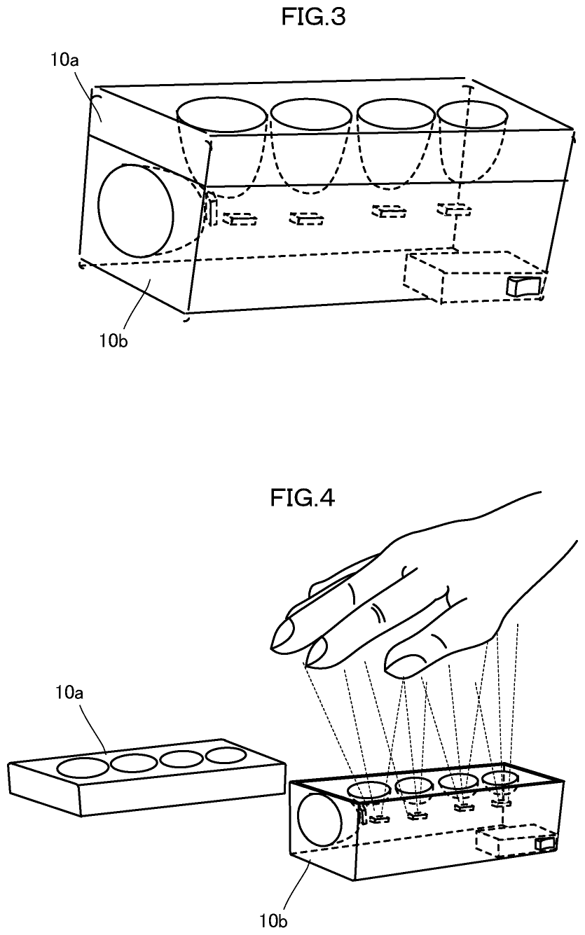

Incidentally, the main body of the portable finger disinfection apparatus 10 may be configured with upper and lower halves which can be opened to expose the inside which the palm can face so that the palm can be disinfected by irradiation of ultraviolet rays. For example, as illustrated in FIG. 3, the main body of the portable finger disinfection apparatus 10 is configured with an upper portion 10a serving as a lid member and a lower portion 10b in which the ultraviolet ray generation device is implemented, and the upper portion 10a and the lower portion 10b can be separated. Then, as illustrated in FIG. 4, when the upper portion 10a is separated and removed to expose the upper side of the lower portion 10b, ultraviolet rays can be emitted upward through the upper side of the ultraviolet ray generation device so that the palm can be disinfected by exposing the palm to the ultraviolet rays as emitted.

### Embodiment 2

In what follows, a portable finger disinfection apparatus in accordance with an embodiment 2 of the present invention will be explained with reference to the accompanying drawings. This portable finger disinfection apparatus makes use of the sterilizing effects of a disinfectant.

FIG. 5 is a perspective view for showing the portable finger disinfection apparatus 30 in accordance with the embodiment 2 of the present invention. The portable finger disinfection apparatus 30 is composed of a rectangular parallelepiped spongy absorbable material which is so soft as to easily be crushed by grasping with a hand, and a nylon cover which is made of a soft nylon material and covering the outer surface of the spongy absorbable material.

The nylon cover is provided with four holes 31, 32, 33 and 34 on the upper surface of the portable finger disinfection apparatus 30, and one hole 35 on the side surface of the portable finger disinfection apparatus 30. Also, in the same manner as the embodiment 1, the spongy material is provided with concaves 31h, 32h, 33h, 34h and 35h into which the forefinger, middle finger, annular finger, pinky finger and thumb can be just fitted respectively through the holes 31, 32, 33, 34 and 35.

Also, inside the main body of the portable finger disinfection apparatus 30, there is a flexible disinfectant tank 40 which is made of a resin and so soft as to easily be crushed by grasping with a hand The flexible disinfectant tank 40 tank stores a disinfectant such as an ethanol solution for disinfection or a chlorous acid water, and serves as a disinfectant supply device. Furthermore, the disinfectant can be replenished through a disinfectant replenishing port 40h.

The disinfectant tank 40 is provided with disinfectant supply tubes 41t, 42t, 43t, 44t and 45t which are projected from this disinfectant tank 40 to supply the disinfectant to the inside of the main body of the portable finger disinfection apparatus 30 from the disinfectant tank 40. The opened tip portions of the disinfectant supply tubes 41t, 42t, 43t, 44t and 45t are arranged to face the bottom portions of the concaves 31h, 32h, 33h, 34h and 35h as disinfectant supply ports for supplying the disinfectant to the bottom portions.

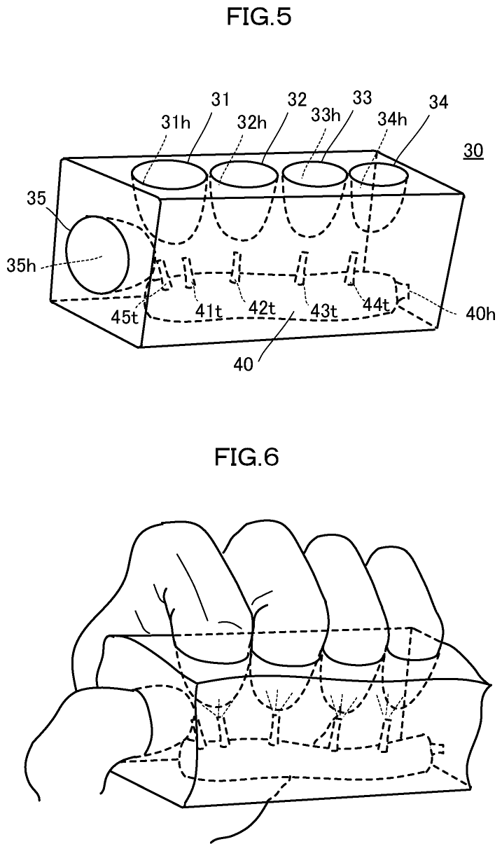

FIG. 6 is a perspective view for showing the usage state of the portable finger disinfection apparatus 30. When this portable finger disinfection apparatus 30 is used, a pressure is applied to the disinfectant tank 40 by inserting the tip portions of the forefinger, middle finger, annular finger, pinky finger and thumb (the parts above the first joints) of a user to the holes 31, 32, 33, 34 and 35, and grasping or pressing with both hands. The disinfectant is then leaked from the opened tip portions of the disinfectant supply tubes 41t, 42t, 43t, 44t and 45t, and oozes out of the concaves 31h, 32h, 33h, 34h and 35h to which the forefinger, middle finger, annular finger, pinky finger and thumb are fitted. It is therefore possible to disinfect the tip portions of the forefinger, middle finger, annular finger, pinky finger and thumb.

Also, as illustrated in FIG. 5 and FIG. 6, the holes 31, 32, 33, 34 and 35, the concaves 31h, 32h, 33h, 34h and 35h, and the disinfectant supply device are symmetrically arranged in the front and back direction as seen from the figures. In other words, it will be understood that this portable finger disinfection apparatus 30 can be used with either of the left and right hands in the same manner.

### Embodiment 3

In what follows, a portable finger disinfection apparatus in accordance with an embodiment 3 of the present invention will be explained with reference to the accompanying drawings. Likewise the embodiment 1, this portable finger disinfection apparatus makes use of the sterilizing effects of ultraviolet rays.

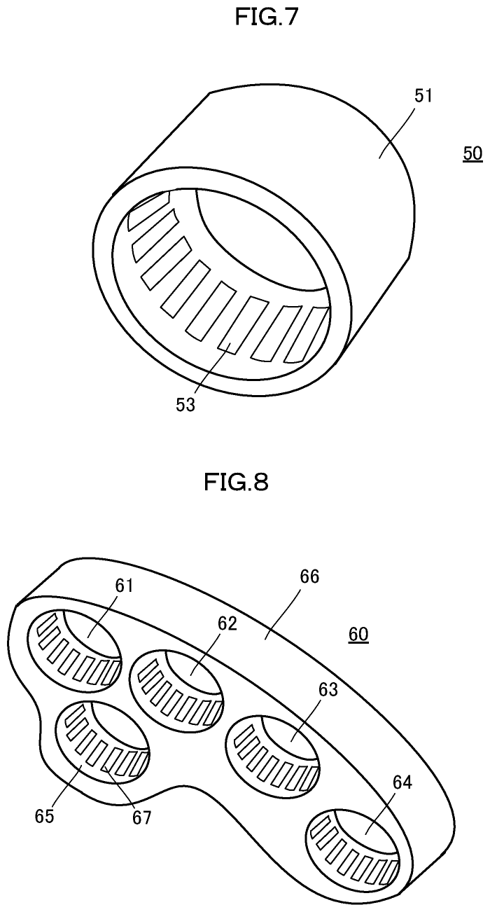

FIG. 7 is a perspective view for showing the portable finger disinfection apparatus 50 in accordance with the embodiment 3 of the present invention. The portable finger disinfection apparatus 50 consists of a ring-like body 51 which is made of a resin and provided with a number of ultraviolet LEDs 53 which are arranged over the inner surface of the ring-like body 51. Although not shown in the figure, a battery, a timer and necessary circuitry are implemented in the ring-like body 51 to supply electric power to the ultraviolet LEDs 53 through wirings to emit ultraviolet rays.

By inserting and sliding the forefinger, middle finger, annular finger, pinky finger and thumb in the ring-like body 51 one after another, it is possible to irradiate and disinfect the entirety of every finger with ultraviolet rays.

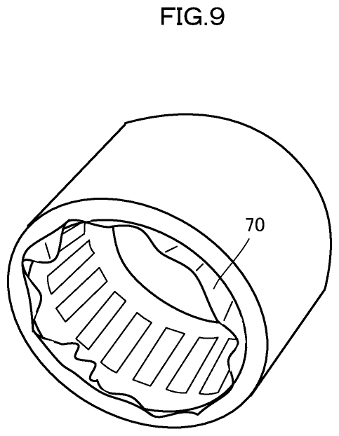

FIG. 8 is a perspective view for showing another example of the portable finger disinfection apparatus. The portable finger disinfection apparatus 60 consists of a resin body 66 in the form of a Brass knuckles provided with five holes 66, 62, 63, 64 and 65 and a number of ultraviolet LEDs 67 which are arranged over the inner surface of each hole. Although not shown in the figure, a battery, a timer and necessary circuitry are implemented in the resin body 66 to supply electric power to the ultraviolet LEDs 67 through wirings to emit ultraviolet rays.

In the case of the portable finger disinfection apparatus 60, the forefinger, middle finger, annular finger, pinky finger and thumb can be inserted together into the holes 61, 62, 63, 64 and 65 respectively to irradiate the five fingers with ultraviolet rays at once.

Meanwhile, in the case of the ring-like body shown in FIG. 7, there is the possibility that ultraviolet rays may leak through the gap between a finger and the ring-like body. To deal with such a problem, a pleat 70 may be provided on the rim of the ring-like body as illustrated in FIG. 9. Such a pleat can be provided also on the rim of each of the holes 66, 62, 63, 64 and 65 of the portable finger disinfection apparatus 60 shown in FIG. 8 to deal with the ultraviolet ray leakage problem.

### Embodiment 4

In what follows, a portable finger disinfection apparatus in accordance with an embodiment 4 of the present invention will be explained with reference to the accompanying drawings. This portable finger disinfection apparatus makes use of both the sterilizing effects of ultraviolet rays and the sterilizing effects of a disinfectant.

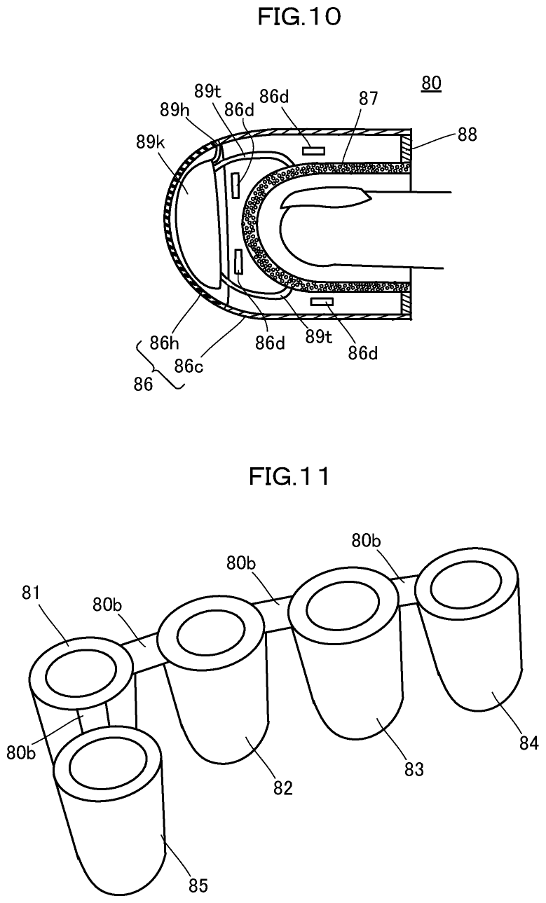

FIG. 10 is a perspective view for showing the portable finger disinfection apparatus 80 in accordance with the embodiment 4 of the present invention. This portable finger disinfection apparatus 80 is in the form of a finger sack into which each fingers is inserted.

Namely, the portable finger disinfection apparatus 80 includes an outer sheath 86 in the form of a finger sack consisting of a cylindrical member 86c and a dome member 86h which is made of a soft resin such as a rubber to close one opening end of the cylindrical member 86c. Furthermore, an inner sheath 87 in the form of a finger sack is installed inside of the outer sheath 86 in order to form a space between the outer sheath 86 and the inner sheath 87. The space between the outer sheath 86 and the inner sheath 87 is closed by a sealing ring 88 at the other opening end of the outer sheath (the cylindrical member 86c).

The inner sheath 87 is made of a spongy absorbable resin material having an ultraviolet ray transparent property such as cellophane, polypropylene or the like. The size of the inner sheath 87 is such that a finger tip can be inserted therein.

A disinfectant tank 89k is installed near the dome member 86h in the space between the outer sheath 86 and the inner sheath 87. The disinfectant tank 89k is used to store a disinfectant such as an ethanol solution for disinfection or a chlorous acid water, and provided with disinfectant supply tubes 89t projecting therefrom. Also, the disinfectant tank 89k is provided with a disinfectant replenishing port 89h for replenishing the disinfectant. One end of each disinfectant supply tube 89t is connected to the disinfectant tank 89k, and the other end is connected to the inner sheath 87. A disinfectant supply device is composed of the disinfectant tank 89k and the disinfectant supply tubes 89t.

The disinfectant tank 89k is made of a soft resin which is deformed by the dome member 86h when the dome member 86h is deformed inward by an external force applied thereto. The disinfectant contained in the deformed disinfectant tank 89k is supplied to the inner sheath 87 through the disinfectant supply tubes 89t. When the disinfectant is supplied from the disinfectant tank 89k, the spongy resin of the inner sheath 87 becomes wet with the disinfectant to disinfect the finger tip inserted into the inner sheath 87. Alternatively, an electric pump can be used to supply the disinfectant in place of application of an external force.

Furthermore, on the inner surface of the cylindrical member 86c of the dome member 86, there are provided a number of ultraviolet LEDs 86d capable of emitting ultraviolet rays toward the inner sheath 87. The ultraviolet LEDs 86d are supplied with electric power from a power supply circuit which is not shown in the figure to emit ultraviolet rays through the inner sheath 87. The ultraviolet rays passing through the inner sheath 87 can disinfect the finger tip inserted into the inner sheath 87. Meanwhile, also in the case of this embodiment, the power supply circuit is provided with a switch and a timer to turn on/off the ultraviolet LEDs 86d.

FIG. 11 is a view for showing an example of a serial assembly of five finger sacks (portable finger disinfection apparatuses 81, 82, 83, 84 and 85) each having the same structure as the portable finger disinfection apparatus 80 shown in FIG. 10. The portable finger disinfection apparatuses 81, 82, 83, 84 and 85 are connected in series with resin belts 80b in order to accommodate the five fingers respectively. In this case, the forefinger, middle finger, annular finger, pinky finger and thumb can be inserted together into the portable finger disinfection apparatuses 81, 82, 83, 84 and 85 respectively to irradiate the five fingers with ultraviolet rays at once. The resin belts 80b have appropriate hardness and flexibility to change the positions and orientations thereof in correspondence with the positions and orientations of the forefinger, middle finger, annular finger, pinky finger and thumb inserted into the portable finger disinfection apparatuses 81, 82, 83, 84 and 85 respectively. Incidentally, the sizes of the portable finger disinfection apparatuses 81, 82, 83, 84 and 85 may be adjusted differently. For example, the portable finger disinfection apparatus 85 to accommodate the thumb may be designed little bigger, and the portable finger disinfection apparatus 84 to accommodate the pinky finger may be designed little smaller.

### Embodiment 5

In what follows, a portable finger disinfection apparatus in accordance with an embodiment 5 of the present invention will be explained with reference to the accompanying drawings.

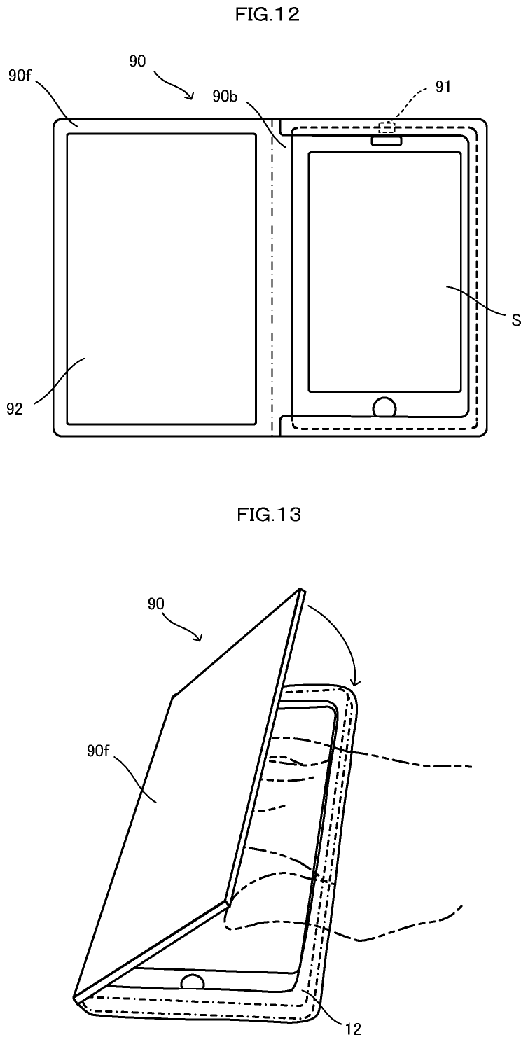

FIG. 12 is a perspective view for showing a smartphone case 90 which is implemented with an ultraviolet ray source so that the smartphone case 90 can be used as a portable finger disinfection apparatus in accordance with the embodiment 5 of the present invention. Like conventional smartphone cases, the smartphone case 90 can be used such that a smartphone S is put or mounted thereon for protection. In addition to this, the smartphone case 90 can be used as a portable finger disinfection apparatus capable of emitting ultraviolet rays.

This smartphone case 90 consists of a back cover 90b on which the smartphone S can be mounted, and a front cover 90f which is pivotally connected to the left edge of the back cover 90b. In the back cover 90b of the smartphone case 90, there are implemented a USB connector 91 which can be connected to the USB port of the smartphone S, and a USB control circuit (not shown in the figure) which can communicate with the smartphone S through the USB connector 91 and the USB port. In the front cover 90f of the smartphone case 90, there is implemented an ultraviolet ray surface emission light 92 which receives electric power from the smartphone S through the USB control circuit.

In addition, an application for controlling the ultraviolet ray surface emission light 92 is installed in the smartphone S. To use the smartphone case 90 as a portable finger disinfection apparatus, electric power is supplied to the ultraviolet ray surface emission light 92 through this application. At this time, an emission time can be set through the application. When this emission time elapses, power supply is automatically halted.

When fingers are exposed to ultraviolet rays, as illustrated in FIG. 13, the fingers are interposed between the ultraviolet ray surface emission light 92 and the smartphone S by placing the fingers on the smartphone S and closing the front cover 90f. By this configuration, the ultraviolet rays can be prevented directly from entering eyes. It is therefore possible to disinfect fingers at any time as long as the smartphone is carried.

### Embodiment 6

In what follows, a portable finger disinfection apparatus in accordance with an embodiment 6 of the present invention will be explained with reference to the accompanying drawings.

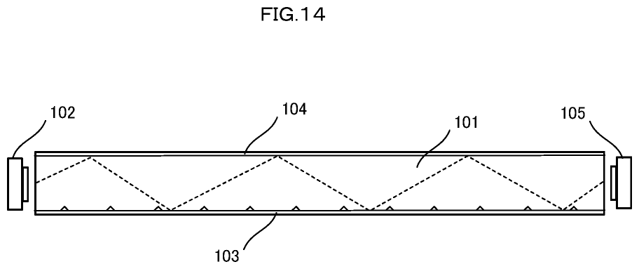

Although the ultraviolet ray source is implemented in the smartphone case according to the embodiment 5, it can be implemented within a smartphone itself. FIG. 14 is a cross sectional view for showing a liquid crystal back light device implemented within a smartphone which functions also as a portable finger disinfection apparatus according to an embodiment 6 of the present invention.

In the same manner as conventional liquid crystal back light devices, this liquid crystal back light device 100 includes a light guide plate 101 made from an acrylic plate, white LEDs 102 provided on one side surface of the light guide plate 101, a reflection sheet 103 provided on the bottom surface of the light guide plate 101 and a diffusion plate 104 provided on the upper surface of the light guide plate 101.

The liquid crystal back light device 100 in accordance with this embodiment provided further with ultraviolet LEDs 105 provided on the opposite side surface of the light guide plate 101 to the one side surface provided with white LEDs 102. When the liquid crystal back light device 100 is used as a light source at the back of a liquid crystal panel (not shown in the figure), only the white LEDs 102 are powered. The white light emitted from the white LEDs 102 enters the light guide plate 101 and is reflected by the surface of the reflection sheet 103 which is processed and diffused by the diffusion plate 104 to generate uniform illumination.

On the other hand, when the liquid crystal back light device 100 is used to perform disinfection with ultraviolet rays, only the ultraviolet LEDs 105 are powered. The ultraviolet rays emitted from the white LEDs 102 enter the light guide plate 101 and are reflected by the surface of the reflection sheet 103 which is processed and diffused by the diffusion plate 104 to generate uniform illumination. In this case, the liquid crystal panel is controlled in its white state to fully transmit the ultraviolet rays which are emitted through the entirety of the screen of the smartphone.

When the smartphone is mounted on a smartphone case, the front cover of the smartphone case can be used to prevent ultraviolet rays directly from entering eyes in the same manner as in the embodiment 5. In the case where no smartphone case is used, finger disinfection can be performed in a pocket of a trouser, a jumper or the like in which a hand is put together with the smartphone which emits ultraviolet rays therein. In this case, ultraviolet ray sterilization can be effectively performed when an ultraviolet ray scattering agent such as titanium dioxide, silicon dioxide or the like is added to the lining of the pocket.

Also in this case, an application for controlling ultraviolet ray emission is installed in the smartphone. At this time, an emission time can be set through the application. When this emission time elapses, power supply is automatically halted. Accordingly, also in accordance with this embodiment, it is possible to disinfect fingers at any time as long as the smartphone is carried.

The portable finger disinfection apparatus in accordance with the present invention can be freely carried by each user so that fingers can easily be disinfected anywhere.

In the case of the embodiments 1 and 2 as described above, disinfection with the ultraviolet rays and disinfection with a disinfectant are described as separate examples. However, the present invention is not limited thereto. For example, both the ultraviolet ray generation device and the disinfectant supply device are implemented within the main body of the portable finger disinfection apparatus to disinfect fingers by the combination of the ultraviolet rays and the disinfectant.

Also, in the case of the embodiments 1 and 2, five holes are provided for the forefinger, middle finger, annular finger, pinky finger and thumb respectively. However, the present invention is not limited thereto. For example, it is possible to provide two holes, i.e., one hole for the thumb and the other hole for the forefinger, middle finger, annular finger and pinky finger. Namely, while the hole for the thumb is as same as in the embodiments 1 and 2, the other hole is provided as an elongated opening such that the forefinger, middle finger, annular finger and pinky finger can be inserted together in the opening. Alternatively, only one hole may be provided for receiving all the five fingers so that gather the forefinger, middle finger, annular finger, pinky finger and thumb can be inserted therein.

The foregoing description of the embodiments has been presented for purposes of illustration and description. It is not intended to be exhaustive or to limit the invention to the precise form described, and obviously many modifications and variations are possible in light of the above teaching. The embodiment was chosen in order to explain most clearly the principles of the invention and its practical application thereby to enable others in the art to utilize most effectively the invention in various embodiments and with various modifications as are suited to the particular use contemplated.

What is claimed is:

1. A portable finger disinfection apparatus comprising:
a main body having a support structure which supports a finger of a user; and
a disinfection mechanism installed in the main body and capable of disinfecting the finger supported by the support structure.

2. The portable finger disinfection apparatus of claim 1 wherein
the support structure is a hole formed on the main body into which a tip portion of the finger of the user can be inserted.

3. The portable finger disinfection apparatus of claim 2 wherein
the disinfection mechanism is an ultraviolet ray generation device which can irradiate the finger inserted into the hole with ultraviolet rays.

4. The portable finger disinfection apparatus of claim 3 wherein
the ultraviolet ray generation device is an ultraviolet ray LED.

5. The portable finger disinfection apparatus of claim 2 wherein
the disinfection mechanism is a disinfectant supply device which can supply the hole with a disinfectant.

6. The portable finger disinfection apparatus of claim 5 wherein
the inside of the main body is filled with a spongy absorbable material which is exposed to the hole and in which the disinfectant supply device is installed, wherein
the disinfectant supply device comprises a flexible disinfectant tank which stores the disinfectant and a disinfectant supply port for supplying the disinfectant to the hole, and wherein
when the user inserts the tip portion of the finger into the hole and applies a pressure to the spongy absorbable material, the flexible disinfectant tank is deformed by the pressure to eject the disinfectant through the disinfectant supply port so that the spongy absorbable material becomes wet with the disinfectant to disinfect the tip portion of the finger tip inserted into the hole.
